(19) United States
(12) Patent Application Publication
(10) Pub. No.: US 2013/0096389 A1
(43) Pub. Date: Apr. 18, 2013

(54) CHAIN OF CUSTODY FOR PHYSIOLOGICAL MONITORING SYSTEM

(71) Applicants: Frank A. Katarow, Pewaukee, WI (US); David A. Uhen, Burlington, WI (US); John Gregory Poulos, Marietta, GA (US); Matthew Blake Oefinger, Sherman, TX (US)

(72) Inventors: Frank A. Katarow, Pewaukee, WI (US); David A. Uhen, Burlington, WI (US); John Gregory Poulos, Marietta, GA (US); Matthew Blake Oefinger, Sherman, TX (US)

(73) Assignee: WATERMARK MEDICAL, LLC, Boca Raton, FL (US)

(21) Appl. No.: 13/271,608

(22) Filed: Oct. 12, 2011

(51) Int. Cl. A61B 5/00

(52) U.S. Cl. 600/300

(57) ABSTRACT

A chain of custody device is provided. The chain of custody device includes a strap for attaching to a body part of a patient. The chain of custody device further includes a receiver connected to the strap and operable to be connected to a physiological monitoring system via a cable. The receiver stores an identifier that is readable by the physiological monitoring system and is usable by the physiological monitoring system to verify that the physiological monitoring system is connected to a correct chain of custody device. The chain of custody device further comprises a security means for preventing undetected removal of the strap and/or receiver. A system comprising the physiological monitoring system and the chain of custody device, and a method for using such system are also described.

### BACKGROUND OF THE INVENTION

1. Field of the Invention

The present invention generally relates to a chain of custody device for use with a physiological monitoring system and systems and methods that utilize the same.

2. Background Art

Sleep apnea is a sleep disorder caused by upper airway obstruction during sleep.

Sleep apnea is associated with frequent awakening. An individual with sleep apnea is rarely aware of having difficulty breathing as a consequence of the airway obstruction, even upon awakening. Symptoms may be present for many years without identification, and the individual may become conditioned to daytime sleepiness and the accompanying fatigue caused by persistent sleep disturbances. Such symptoms may have a negative impact on the cognition of the individual in the waking state by causing, for example, impaired reaction time, vision problems, difficulty with information processing, impaired judgment, and short term memory loss. Sleep apnea may also affect the behavior of the individual by causing, for example, reduced vigilance, reduced motivation, moodiness, and aggressiveness. The combination of negative cognitive and behavioral effects can be very dangerous in many occupations, especially ones that require operation of machinery or manual labor.

Sleep apnea may be diagnosed by a sleep study, called polysomnography (PSG). However, a PSG can cost up to $2,000 as it is administered and analyzed by a trained technician and a Board Certified Sleep Specialist. A PSG also requires a patient to be housed and monitored overnight at a sleep center. Such sleep centers are limited in number. The lack of available sleep centers along with the high cost of the PSG cause the PSG to be an inconvenience for many patients.

Alternatively, a home sleep study system that collects and analyzes physiological signals may be used for the diagnosis or treatment of sleep apnea. An example of a home sleep study system is the ARES™ Unicorder provided by Watermark Medical of Boca Raton, Fla. The home sleep study system is a portable system that allows a patient to be monitored away from a clinical setting. This portable system is provided to the patient who wears the system during normal sleep in his own home. The patient then returns the system to an office of a clinician, where data may be extracted from the system for analysis.

While the home sleep study is less expensive and more convenient for the patient than the PSG, it poses a problem of data authenticity. Individuals whose livelihoods depend on the result of a sleep study, for example, those who operate machinery or drive for prolonged periods of time for a living, may be motivated to falsify data collected from a home sleep study in order to be allowed to work. In a PSG, the sleep study is monitored by trained professionals, and there is no risk of a patient providing false data. In a home sleep study, a patient may persuade someone else to wear the home sleep study system and undergo the home sleep study instead of the patient. In this case, data collected by the system would pertain to someone else and not the patient. A clinician would not be able to determine that the system had been defeated and has no means to prevent or detect such tampering act.

What is needed, then, is a system, method, and device that enables a professional to verify the authenticity of data collected by a home sleep study system and to prevent undetected tampering with such a system. The desired system, method, and device should be able to establish a chain of custody of data collected from a home sleep study ensuring that the home sleep study remains a viable option for diagnosis and treatment of sleep apnea. The desired system, method, and device should also be applicable to other portable physiological monitoring systems in addition to those used for home sleep studies.

### BRIEF SUMMARY OF THE INVENTION

The present invention provides a means by which a chain of custody may be established for data collected by a portable physiological monitoring system, including but not limited to a home sleep study system. The chain of custody may be used, for example, to guarantee that data collected by a home sleep study system is from an intended patient rather than some other person.

In particular, a chain of custody device is described herein. The chain of custody device comprises a strap for attaching to a body part of a patient. The chain of custody device further comprises a receiver connected to the strap. The receiver is operable to be connected to a physiological monitoring system via a cable. The receiver stores an identifier that is readable by the physiological monitoring system and is usable by the physiological monitoring system to verify that the physiological monitoring system is connected to a correct chain of custody device. The chain of custody device further comprises a security means for preventing undetected removal of the strap and/or the receiver.

Also described herein is a system that includes a physiological monitoring system and a chain of custody device. The chain of custody device comprises a strap for attaching to a body part of a patient and a receiver connected thereto. The receiver is operable to be connected to the physiological monitoring system via a cable. The receiver stores an identifier that is readable by the physiological monitoring system and usable thereby to verify that the physiological monitoring system is connected to a correct chain of custody device. The chain of custody device further comprises a security means for preventing undetected removal of the strap and/or the receiver.

A method for using a home sleep study system in accordance with an embodiment of the present invention is also described herein. The method comprises attaching a strap of a chain of custody device to a body part of a patient. The chain of custody device includes the strap and a receiver that is operable to be connected to a physiological monitoring system via a cable. The receiver stores an identifier that is readable by the physiological monitoring system and is usable thereby to verify that the physiological monitoring system is connected to the correct chain of custody device. The chain of custody device also includes a security means for preventing the undetected removal of the strap and/or the receiver. The method further comprises securing the cable to the physiological monitoring system via a security label, and providing the patient with the physiological monitoring system.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the relevant art(s) to make and use the invention.

FIG. 1 illustrates physical components of a chain of custody device in accordance with an embodiment of the present invention.

FIG. 2 illustrates a strap of a chain of custody device in an unfastened state in accordance with an embodiment of the present invention that utilizes a hook and loop fastener.

FIG. 3 illustrates a strap of the chain of custody device in a fastened state in accordance with an embodiment of the present invention that utilizes a snap rivet fastener.

FIG. 4 illustrates a security tag of a chain of custody device in accordance with an embodiment of the present invention.

FIG. 5 illustrates a security tag secured on a strap of a chain of custody device in accordance with an embodiment of the present invention.

FIG. 6 illustrates a cable connected to a receiver of a chain of custody device in accordance with an embodiment of the present invention.

FIG. 7 illustrates a cable that may be connected to a receiver of a chain of custody device in accordance with an embodiment of the present invention.

FIG. 8 provides an exploded view of a receiver of a chain of custody device in accordance with an embodiment of the present invention.

FIG. 9 illustrates a layout of a printed circuit board (PCB) of a receiver of a chain of custody device in accordance with an embodiment of the present invention.

FIG. 10 illustrates a system comprising a chain of custody device and a physiological monitoring system in accordance with an embodiment of the present invention.

FIG. 11 illustrates a flowchart of a method for using a chain of custody device in conjunction with a physiological monitoring system in accordance with an embodiment of the present invention.

FIG. 12 illustrates a security label of a system in accordance with an embodiment of the present invention.

FIGS. 13A and 13B illustrate the security label securing a cable to the system in accordance with an embodiment of the present invention.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

### A. INTRODUCTION

A chain of custody device is described herein that facilitates the integrity of data collected by a portable physiological monitoring system, such as a home sleep study system. The chain of custody device ensures that data collected by the physiological monitoring system is collected only from an intended patient and deters the intended patient from tampering with the collection process and/or equipment. A system that includes the chain of custody device and the physiological monitoring system as well as a method for using the same are also described herein.

In at least one embodiment, the chain of custody device includes a strap and a receiver connected to the strap. The receiver is operable to be connected to a physiological monitoring system via a cable and stores an identifier that is readable by the physiological monitoring system and usable thereby to verify that the physiological monitoring system is connected to the appropriate chain of custody device.

To prepare for a study, such as a home sleep study, a clinician may cause the same identifier that is stored on the receiver of the chain of custody device to be stored in memory that is internal to or otherwise accessible to the physiological monitoring system, thereby creating an association between the chain of custody device and the physiological monitoring system. The clinician may then attach the strap of the chain of custody device to a body part of a patient. For example, the clinician may attach the strap to the neck of the patient while the patient is at an office of the clinician. The patient may be instructed not to remove the strap.

Before the patient goes to sleep, the patient connects the receiver of the chain of custody device to the physiological monitoring system via a cable to establish a communication link between the physiological monitoring system and the receiver. If this connection is not made or is otherwise impaired, the physiological monitoring system will cease collecting physiological data and alert the patient to the problem with an audible alarm or other perceptible warning. Furthermore, the physiological monitoring system uses the communication link to obtain the identifier stored on the receiver and determine whether the obtained identifier matches the identifier stored in its own memory. If the identifier obtained from the receiver does not match the identifier stored in memory by the physiological monitoring system, then the physiological monitoring system will cease collecting physiological data and alert the patient to the problem with an audible alarm or other perceptible warning.

When the patient returns to the office of the clinician, the strap may be removed by the clinician and inspected for signs of tampering. If signs of tampering are detected then the result of the home study may be void at the discretion of the clinician. Thus, the chain of custody device allows data collection only from the intended patient.

Various features may be used to deter the patient from tampering with the aforementioned chain of custody device. In an example embodiment described below, the chain of custody device includes a security means comprising a security tag that is sealed over a fastener of the strap. In another example embodiment, the security means includes a snap rivet with a permanent one-way fastening feature. Such security means make it difficult for the patient to remove the strap without leaving behind evidence of tampering. In a further example embodiment, the physiological monitoring system is capable of detecting the use of an unauthorized cable to connect the chain of custody device to the physiological monitoring system. Still further, a clinician may connect one end of an authorized cable to the physiological monitoring system and then affix the authorized cable to the physiological monitoring system using a security label(s), thereby making it difficult for the patient to disconnect the authorized cable from the physiological monitoring system without leaving behind evidence of tampering. In a still further embodiment, a cable may be provided that is only long enough to enable the physiological monitoring system to be connected to the appropriate body part of the patient wearing the chain of custody device. However, the invention is not limited to these anti-tampering features. Persons skilled in the relevant art(s) will readily appreciate that other anti-tampering features may accomplish a same or similar purpose. For example, a cable with custom connectors may be utilized to prevent the usage of an unauthorized, commercially-available cable because such cables would not have the custom connectors to properly connect to the physiological monitoring system and the chain of custody device.

The present invention is not limited to only a home sleep study. Rather, other studies may benefit from the chain of custody device, including studies that do not involve data collection or sleeping at home. The chain of custody device may be used with any system or device worn by a patient to monitor any type of physical activity that requires verification that data collected by the system or device was acquired from that specific patient. Thus, for instance, the chain of custody device may be used with a system for managing medical treatment. Moreover, such system or device may be attached to any suitable body part of the patient, such as the neck, the arm, or the wrist.

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

### B. EXAMPLE CHAIN OF CUSTODY DEVICE IN ACCORDANCE WITH AN EMBODIMENT OF THE PRESENT INVENTION

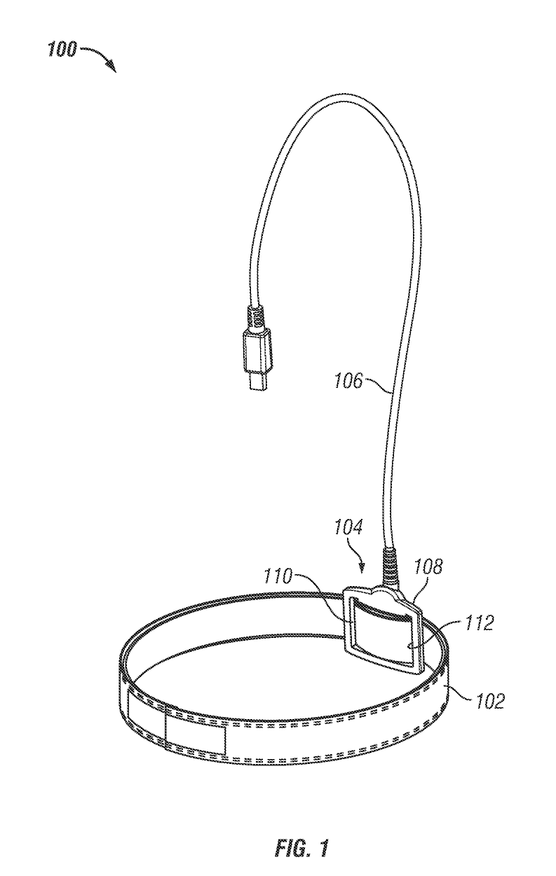

FIG. 1 shows an example of a chain of custody device 100 in accordance with an embodiment. As shown in FIG. 1, chain of custody device 100 includes a strap 102 and a receiver 104 connected thereto. Strap 102 may be attached to a neck of a patient by wrapping strap 102 around the neck and fastening it. For ease of illustration, strap 102 may be depicted and described herein as being a neck strap or collar; however the invention is not so limited to this embodiment. Strap 102 may be attached to any suitable body part of a patient, for example, the neck, the arm, or the wrist.

FIG. 1 shows strap 102 in a fastened state. Strap 102 may be attached to the patient by a clinician in an office to facilitate a study, such as a home sleep study. After the patient leaves the office, the patient is required to wear strap 102 at all times until strap 102 is removed by the clinician at the conclusion of the study. To begin the study, the patient connects a physiological monitoring system to receiver 104 on strap 102 via a cable 106. In an embodiment in which the physiological monitoring system is a home sleep study system, the patient may connect the physiological monitoring system to receiver 104 before going to sleep. As will be discussed below, the physiological monitoring system will only perform a data collection function when properly connected to receiver 104. Chain of custody device 100 is used to ensure that data collected during the study is collected only from an intended patient and not from any other person. Chain of custody device 100 includes features that make it difficult to defeat this purpose of chain of custody device 100 without leaving behind evidence of tampering. Such evidence of tampering may be detected by the clinician, who may decide to void the results of the study. Thus, patients may be less motivated to tamper with chain of custody device 100 to avoid the undesirable consequence of having the results voided. Further details concerning each component of chain of custody device 100 will now be described.

In an embodiment, receiver 104 comprises a housing 108 that includes an integrated circuit (IC) and an interface to which cable 106 may be connected. The IC includes a memory that stores a unique serial number. The unique serial number is readable by a physiological monitoring system when the physiological monitoring system is connected to receiver 104 via cable 106.

Strap 102 may be formed from any of a variety of cloth, organic, or synthetic materials. For example, strap 102 may be formed from a material that is determined to be comfortable when worn around the body part of a patient, the neck, for example. In one embodiment, strap 102 comprises VELTEX®, which is a material that includes a napped cloth surface on one side and a nylon tricot surface on the other. In addition to providing comfort, VELTEX® offers various advantages over other materials including flexibility, strength, and low cost. Alternatively, strap 102 may be formed from a soft plastic or a synthetic material made of polyethylene fibers.

In the embodiment shown in FIG. 1, receiver 104 is designed such that it can be easily connected to or removed from strap 102 when strap 102 is in an unfastened state. In accordance with this embodiment, the connection between receiver 104 and strap 102 is formed by inserting strap 102 through both a first opening 110 and a second opening 112 in housing 108 of receiver 104 when strap 102 is in the unfastened state, wherein first opening 110 and second opening 112 are on opposite ends of housing 108. In further accordance with this embodiment, when strap 102 is fastened, receiver 104 cannot be removed therefrom without unfastening or breaking strap 102. Making receiver 104 removable from strap 102 may be desirable in an embodiment in which strap 102 is disposed of after each study but receiver 104 is reused.

In an alternate embodiment, other means may be used to removably connect receiver 104 to strap 102. In still further embodiments, receiver 104 may be integrated with strap 102 such that receiver 104 cannot easily be removed therefrom in either a fastened or unfastened state.

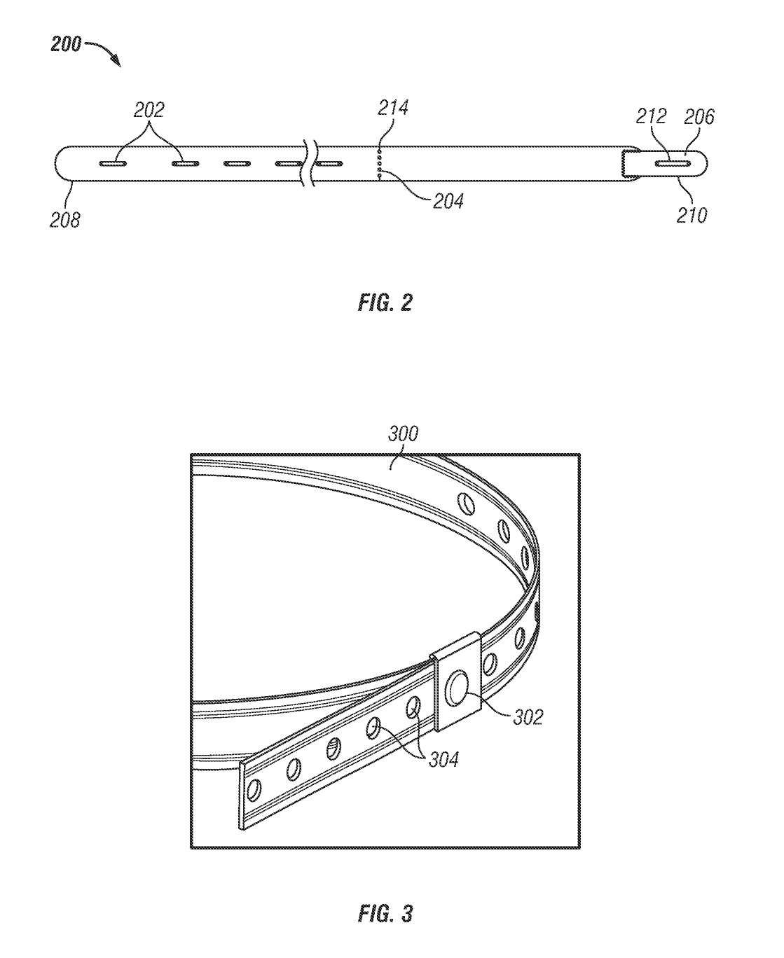

FIG. 2 depicts a strap 200 of a chain of custody device in accordance with an example embodiment. As shown in FIG. 2, strap 200 includes a first end 208, a second end 210, a fastener 206, a plurality of openings 202 at first end 208, and an opening 212 in fastener 206.

In one embodiment, the surface of strap 200 shown in FIG. 2 (also referred to herein as the “near side” of strap 200) comprises a napped cloth surface while the opposing surface (also referred to herein as the “far side” of strap 200) comprises a nylon tricot surface. In further accordance with such an embodiment, strap 200 may be fastened to the neck of a patient such that the nylon tricot surface faces toward the body of the patient while the napped cloth surface faces away from the body of the patient. In still further accordance with such an embodiment, the surface of fastener 206 that is not shown in FIG. 2 (also referred to herein as the “far side” of fastener 206) may comprise a plurality of VELCRO hooks, and the process of fastening strap 200 to a neck of a patient may comprise bringing first end 208 and second end 210 of strap 200 together such that the VELCRO hooks on the far side of fastener 206 become engaged with the napped cloth surface on the near side of strap 200. The point of engagement may be selected to determine the size of the loop formed by strap 200 when in a fastened state, thereby enabling strap 200 to accommodate different neck sizes.

As a further part of fastening strap 200, opening 212 in fastener 206 may be selectively aligned with one of openings 202 at first end 208, and a sealable security tag may be inserted through the aligned openings to prevent unauthorized removal of strap 200. Such selective alignment of opening 212 with any of openings 202 further enables strap 200 to be fastened in a manner that accommodates different neck sizes. For example, larger neck sizes may be accommodated when opening 212 is aligned with openings 202 closer to first end 208 while smaller neck sizes may be accommodated when opening 212 is aligned with openings 202 closer to the middle of strap 200.

For smaller neck sizes, strap 200 may be longer than necessary. In such an event, excess material of strap 200 may be cut from first end 208 to make strap 200 shorter. While it is important to size strap 200 to be as comfortable as possible for a patient, it is necessary for strap 200 to be tight enough such that strap 200 cannot be slipped over the head of the patient. In an embodiment, strap 200 may be 24 inches in length. However, as will be readily appreciated by persons skilled in the relevant art(s), strap 200 may be of any other length suitable for a particular implementation.

As shown in FIG. 2, openings 202 comprise narrow slits approximately 0.75 inches in length that are 1.25 inches apart, while opening 212 comprises a narrow slit approximately 1.0 inch in length. However, openings 202 and opening 212 are not limited to these shapes and sizes. Rather, openings 202 and opening 212 may be of any shape and size, such as rectangular, circular, elliptical holes or slits. Moreover, as long as the integrity of strap 200 is preserved--that is, strap 200 must be strong enough to hold a receiver without breaking--there may be any number of openings present on strap 200. The openings may be formed by any tool or process known in the relevant art(s). For example, a die cutting process may be used to create the openings to seal the edges and prevent fraying.

As further shown in FIG. 2, strap 200 further includes a perforated region 204. Perforated region 204 is a safety feature that forms a breakage region in strap 200. Perforated region 204 facilitates the breaking apart of strap 200 at the breakage region when stretched. Such safety feature may be useful in case of an emergency, to prevent a patient wearing strap 200 from harm should strap 200 become too tight (e.g., due to swelling of the neck of the patient, or strap 200 becoming caught on an object). Perforated region 204 may also serve as a means for preventing undetected removal of strap 200. For example, if a patient attempts to pull strap 200 over his head, strap 200 may break apart at the breakage region. Such a tear in strap 200 would be detectable by a clinician upon inspection of strap 200. Perforated region 204 may comprise of a series of holes in one or more lines that form the breakage region, which may be weaker in strength than any other portion of strap 200. Furthermore, as shown in FIG. 2, a small slit 214 may be formed between an edge of strap 200 and a hole to further facilitate tearing or breaking of strap 200. Perforated region 204 may be created with a die cut, for example, although other tools or processes may also be used.

As noted above, fastener 206 may be used to fasten first end 208 and second end 210 of strap 200 around a body part of a patient. Although fastener 206 is described above as comprising a hook-and-loop fastener, fastener 206 may comprise any suitable means for securing first end 208 and second end 210 of strap 200 together. For example, FIG. 3 illustrates a strap 300 in accordance with an alternate embodiment that utilizes a snap rivet 302 as a fastener. As will be appreciated by persons skilled in the art, snap rivet 302 may comprise two flat panels that can be securely connected to each other via corresponding mating parts that are attached thereto. The connection of the flat panels may be made through any one of a number of holes 304 in strap 300, thereby allowing strap 300 to be fastened in a manner that accommodates various neck sizes. As another example, instead of a hook-and-loop fastener, strap 200 may be secured by bringing first end 208 and second end 210 together such that opening 212 is aligned with one of openings 202. A tag with an adhesive portion may be inserted through the aligned openings and the ends thereof may be sealed together to fasten strap 200. Still other fastening mechanisms may be used.

Returning now to the description of FIG. 1, chain of custody device 100 may also include a security means for preventing undetected removal of strap 102 and/or for preventing undetected removal of receiver 104 from strap 102. Such security means may help to ensure the integrity of data collected during a home study. For example, the security means may make it difficult to defeat the chain of custody device without damaging some part thereof. Thus, evidence of damage would be detectable by a clinician who is administering the home study. Therefore, with the security means in place, a patient would be deterred from tampering with chain of custody device 100, particularly with strap 102. Some implementations of the security means are described below in accordance with certain embodiments of the present invention. However, the present invention is not so limited to these implementations.

In the embodiment shown in FIG. 3, the security means may comprise snap rivet 302, which may include a permanent one-way feature that prevents strap 300 from becoming unfastened without evidence of damage to snap rivet 302 or strap 300. Snap rivet 302 may thus serve as both a fastener and a security means.

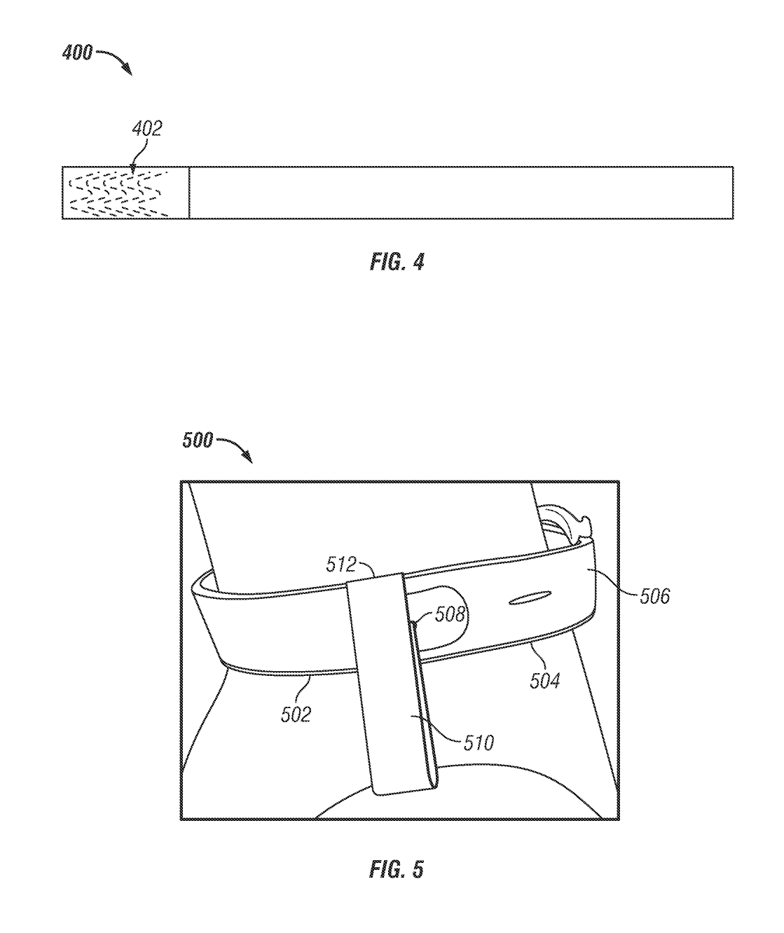

In another embodiment, the security means comprises a security tag 400 as shown in FIG. 4. Security tag 400 may be used to prevent hoop and loop fastener 206, depicted in FIG. 2, from being unfastened in an unauthorized manner. Security tag 400 may comprise, for example, a strong synthetic material that is difficult to tear, such as TYVEK®. As shown in FIG. 4, security tag 400 includes an adhesive portion 402. Adhesive portion 402 may be covered by a release liner that may be peeled away to expose adhesive portion 402. Referring back to FIG. 2, strap 200 includes first end 208 having openings 202 and fastener 206 having opening 212. When opening 212 is aligned with one of openings 202, security tag 400 may be passed through the aligned openings and then the ends thereof may be sealed together to form a loop using adhesive portion 402.

FIG. 5 illustrates a further example of a chain of custody device 500 that is fastened using such a security tag 510. As shown in FIG. 5, when a strap 506 of chain of custody device 500 is in a fastened state, openings at ends 502 and 504 of strap 506 overlap to form an overlapped opening 508. A security tag 510 may be inserted through overlapped opening 508. When a release liner that covers an adhesive portion 512 of security tag 510 is removed, the exposed adhesive portion 512 may be used to seal opposing ends of security tag 510 together, thereby forming a sealed loop that secures ends 502 and 504 of strap 506 together. Thus, in accordance with the embodiment shown in FIG. 5, it is not possible to unfasten a hook and loop fastener of strap 506 without damaging security tag 510.

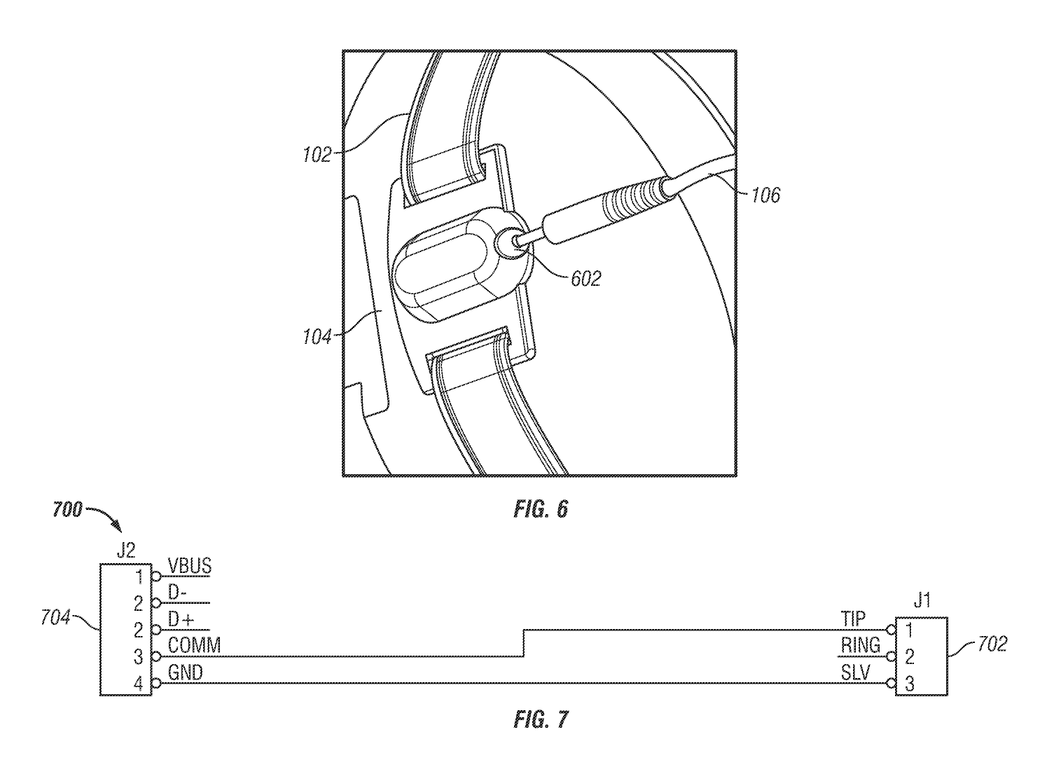

FIG. 6 provides a further view of receiver 104 of FIG. 1, which is connected to strap 102 and may be connected to a physiological monitoring system via a cable 106. As shown in FIG. 6, cable 106 may be connected to receiver 104 via a connector 602. In one embodiment connector 602 comprises an audio jack. As will be appreciated by person skilled in the relevant art(s), an audio jack may also be referred to as a TRS (tip, ring, sleeve) connector, a mini-stereo, stereo plug, mini-jack, headphone jack, or phone jack or plug.

FIG. 7 illustrates a cable 700, which is one example implementation of cable 106. As shown in FIG. 7, cable 700 includes a first connector 702 in the form of an audio jack on one end and a second connector 704 in the form of a micro-USB connector on the other end. Second connector 704 may comprise, for example, a standard Mini-B connector. First connector 702 may be used to connect to a receiver, such as receiver 104, while second connector 704 may be used to connect to a physiological monitoring system.

As further shown in FIG. 7, cable 700 is configured such that a TIP conductor of first connector 702 is connected to a COMM conductor of second connector 704 and a SLV conductor of first connector 702 is connected to a GND conductor of second connector 704. These two connections may facilitate the use of a serial data communication protocol to transfer data between receiver 104 and a physiological monitoring system. The RING conductor of first connector 702 is left open and unused. In one embodiment, the physiological monitoring system may be configured to detect if an unauthorized cable, for instance, an extension cable or a substitute cable, that does not include such an internal configuration has been used to connect receiver 104 thereto and to generate an alarm and/or cease collecting data in response to such detection. In another embodiment, receiver 104 or both the receiver 104 and the physiological monitoring system may be configured to detect such unauthorized cable. For example, a patient seeking to defeat chain of custody device 100 may try to substitute a longer, commercially-available cable for cable 106 to enable data to be collected from a person other than himself The longer, substitute cable may attempt to utilize the RING conductor, and such attempt may be detectable by the physiological monitoring system and/or receiver 104. In a similar manner, the patient may also seek to defeat chain of custody device 100 by adding an extension cable to lengthen cable 106. The extension cable may attempt to utilize the RING conductor, and such attempt may be detectable by the physiological monitoring system and/or receiver 104. By detecting the use of such unauthorized cable, the physiological monitoring system and/or the receiver can provide a further measure for ensuring the integrity of the data collected from a patient.

Although cable 700 is shown as including an audio jack and micro-USB connector, persons skilled in the relevant art(s) will appreciate that other standard or custom connectors may be used to connect receiver 104 to cable 106 and to connect cable 106 to a physiological monitoring device. The length of cable 700 may be selected so as to make it difficult to collect physiological data from anyone other than an intended patient. For example, in an embodiment in which the physiological monitoring system is worn on the forehead of the patient, the length of cable 700 may be selected to be 19 inches. At this length, cable 700 would be just long enough to connect receiver 104 attached to the neck of the patient to the physiological monitoring system worn on the forehead of that patient. The limited length of cable 700 would make it difficult and uncomfortable to connect receiver 104 attached to the neck of the patient to the physiological monitoring system if it were attached to the forehead of anyone other than the patient. Persons skilled in the relevant art(s) will readily appreciate that other cable lengths may be used depending on the location of the physiological monitoring system in relation to the location of the receiver or other factors, such as physical characteristics of the patient.

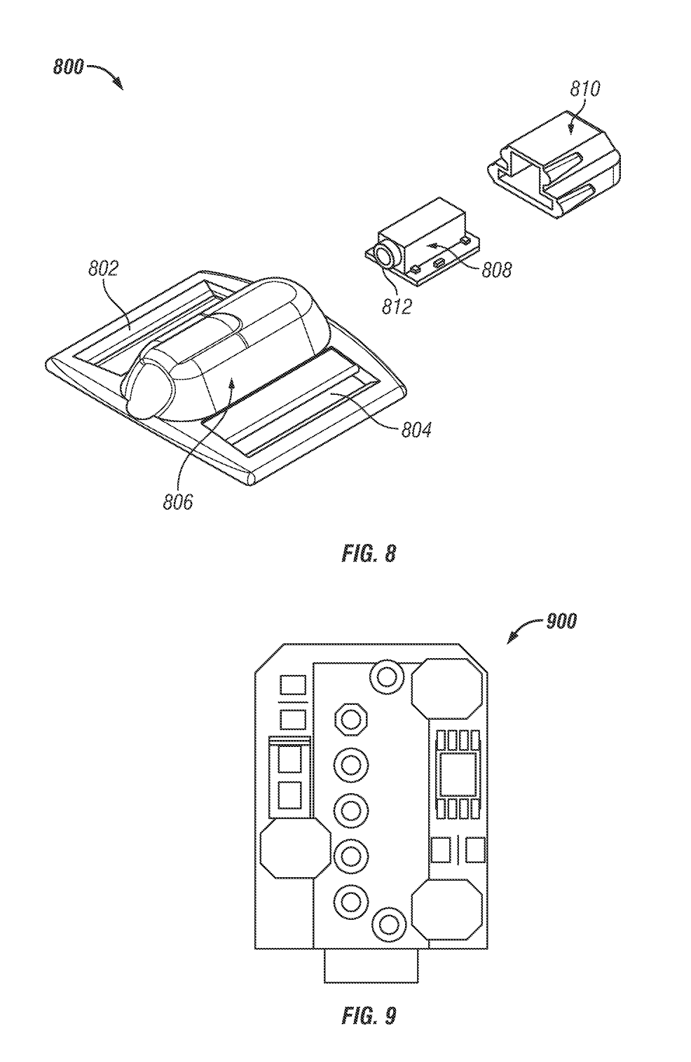

FIG. 8 provides an exploded view of a receiver 800, which comprises one example implementation of receiver 104 of FIG. 1. As shown in FIG. 8, receiver 800 includes a printed circuit board (PCB) assembly 808 having an audio jack socket 812. PCB assembly 808 also houses an integrated circuit that stores a unique identifier. Such unique identifier may be read by a physiological monitoring system that is connected to audio jack socket 812 via a cable, such as cable 106. An example layout 900 of PCB assembly 808 is shown in FIG. 9.

As further shown in FIG. 8, PCB assembly 808 is covered by a mold shield 810 and is ultimately protected by overmold 806. Mold shield 810 protects PCB assembly 808 from mold heat and may comprise, among others, a plastic material. Overmold 806 includes a first opening 802 and a second opening 804 to enable receiver 800 to be removably connected to a strap, such as strap 102 depicted in FIG. 1. First opening 802 and second opening 804 enable receiver 800 to be slipped on or off strap 102 such that strap 102 may be discarded after one use, but receiver 800 may be used multiple times.

### C. EXAMPLE SYSTEM COMPRISING A CHAIN OF CUSTODY DEVICE IN ACCORDANCE WITH AN EMBODIMENT OF THE PRESENT INVENTION

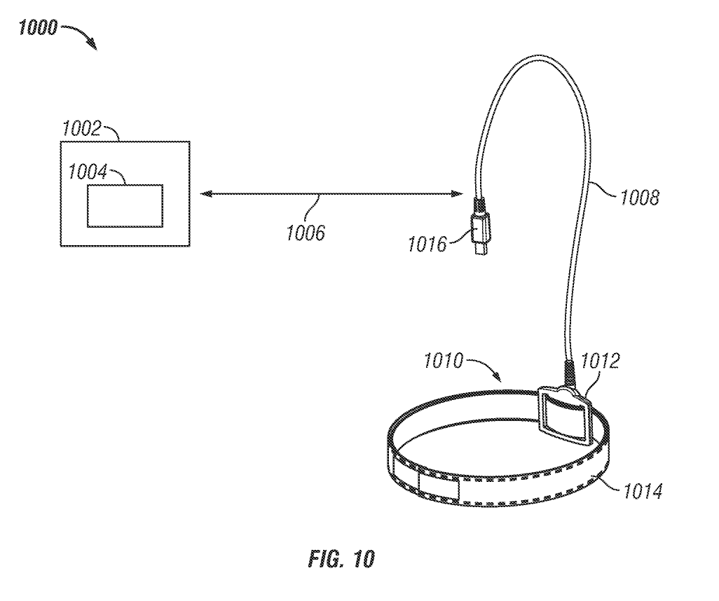

FIG. 10 illustrates a system 1000 that includes a physiological monitoring system 1002 and a chain of custody device 1010 in accordance with an embodiment. Chain of custody device 1010 may be similar to or the same as chain of custody device 100 as described above in reference to FIG. 1. Therefore, all the details discussed above with respect to example chain of custody device 100 may also be present in chain of custody device 1010.

System 1000 facilitates the collection of physiological data from a patient using physiological monitoring system 1002 while chain of custody device 1010 ensures the authenticity of data collected by physiological monitoring system 1002 (i.e., it ensures that such data is collected from the patient and not any other person). As shown in FIG. 10, chain of custody device 1010 is connected to physiological monitoring system 1002 via a cable 1008, which may be the same as or similar to cable 106 as described above in reference to FIG. 1. Receiver 1012 stores an identifier that is readable by physiological monitoring system 1002. The identifier is usable by physiological monitoring system 1002 to verify that physiological monitoring system 1002 is connected to a correct chain of custody device. In one embodiment, receiver 1012 draws power and communicates with physiological monitoring system 1002 by using a one-wire serial data protocol, although other protocols and communication links may be used.

System 1000 functions in an intended manner only after appropriate formatting of physiological monitoring system 1002. Following or during the formatting of physiological monitoring system 1002, the identifier from receiver 1012 of chain of custody device 1010 is collected and stored in a data storage device integrated with or otherwise accessible to physiological monitoring system 1002 such as memory 1004. Memory 1004 may include one or more of volatile or non-volatile, removable and non-removable memory accessible to physiological monitoring system 1002. For example, memory 1004 may include but is not limited to one or more of random access memory (RAM), read only memory (ROM), EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store desired information and which may be accessed by physiological monitoring system 1002.

When physiological monitoring system 1002 is activated to collect physiological data from a patient, physiological monitoring system 1002 polls receiver 1012 periodically for the identifier stored thereon. Polling of receiver 1012 may occur, for instance, every two seconds, to minimize the power drain on physiological monitoring system 1002, which may operate on battery power. If physiological monitoring system 1002 obtains the identifier from receiver 1012 and verifies that the identifier obtained from receiver 1012 matches the identifier stored in memory 1004, then connection 1006 between physiological monitoring system 1002 and chain of custody device 1010 is determined to be valid and physiological monitoring system 1002 collects physiological data from a patient. However, if physiological monitoring system 1002 cannot obtain an identifier from receiver 1012 or does not receive the proper identifier from receiver 1012, then physiological monitoring system 1002 will cease collection of physiological data from the patient and/or generate an alarm to alert the patient to the problem.

The alarm generated by physiological monitoring system 1002 may be, for example, an audible alarm, a visual alarm, or any other type of alarm that is perceptible to the patient. With respect to a visual alarm, physiological monitoring may utilize, for example, a light or light-emitting diode (LED) that flashes to indicate a problem. Any number of problems may trigger an alarm. Some example problems that may result in an impaired connection between physiological monitoring system 1002 and receiver 1012 may be when physiological monitoring system 1002 expects to obtain one identifier from receiver 1012 but actually fails to read the expected identifier or reads another identifier, when receiver 1012 or physiological monitoring system 1002 detects a presence of an unauthorized cable that is not intended to be used with chain of custody device 1010, or when a patient initiates data collection without securely or properly connecting cable 1008 to receiver 1012.

In an embodiment, physiological monitoring system 1002 may be an Apnea Risk Evaluation System (ARES) as described in U.S. Pat. No. 6,811,538 entitled “Sleep Apnea Risk Evaluation” by Westbrook et al., filed Dec. 28, 2001 and U.S. Pat. No. 7,297,119 entitled “ Sleep Apnea Risk Evaluation” also by Westbrook et al., filed Aug. 25, 2004. The disclosures of these patents are hereby incorporated by reference.

### D. EXAMPLE METHOD OF USING A CHAIN OF CUSTODY DEVICE IN CONJUNCTION WITH A PHYSIOLOGICAL MONITORING SYSTEM IN ACCORDANCE WITH AN EMBODIMENT OF THE PRESENT INVENTION

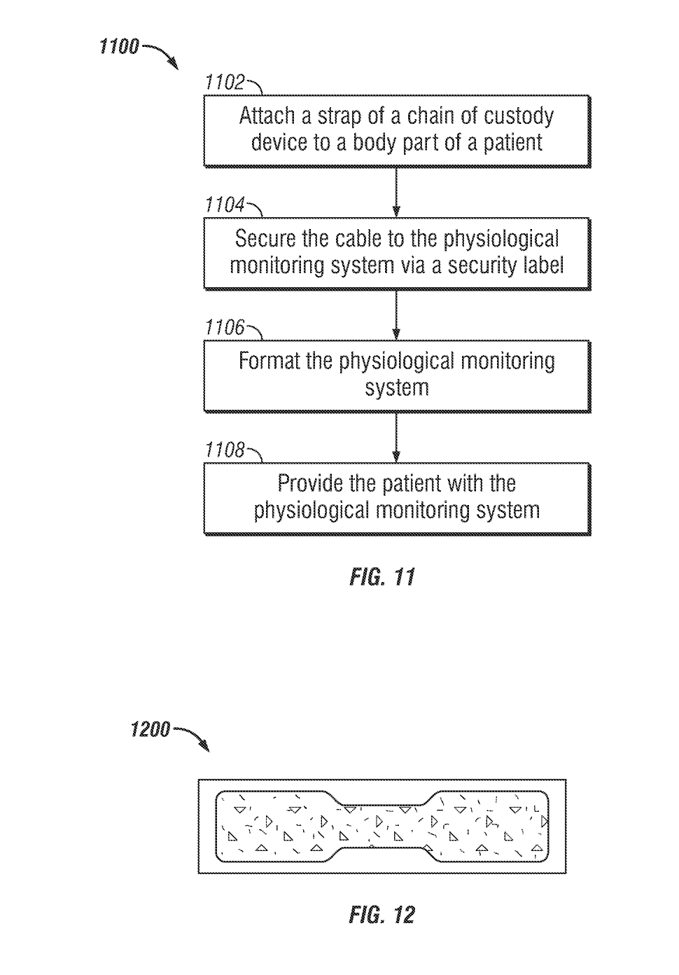

A method for using a chain of custody device in conjunction with a physiological monitoring system accordance with an example embodiment will now be described. In particular, the method will be described in reference to flowchart 1100 of FIG. 11. The method will be described with continued reference to example chain of custody device 100 and system 1000 described above at least in reference to FIGS. 1 and 10, although the method is not so limited to those embodiments.

As shown in FIG. 11, the method of flowchart 1100 begins at step 1102, in which a strap of a chain of custody device is attached to a body part of a patient. In system 1100, this step may be performed by wrapping a strap 1014 of chain of custody device 1010 around a neck of a patient and fastening it, thereby attaching chain of custody device 1010 to the patient.

At step 1104, a cable for connecting a chain of custody device to a physiological monitoring system is secured to the physiological monitoring system via a security label. In system 1000, cable 1008 may be used to connect chain of custody device 1010 to physiological monitoring system 1002. Cable 1008 must be physically and/or electrically connected to both devices to form a valid connection 1006. Once cable 1008 is properly connected, a security label may be used to secure cable 1008 to physiological monitoring system 1002 in accordance with step 1104.

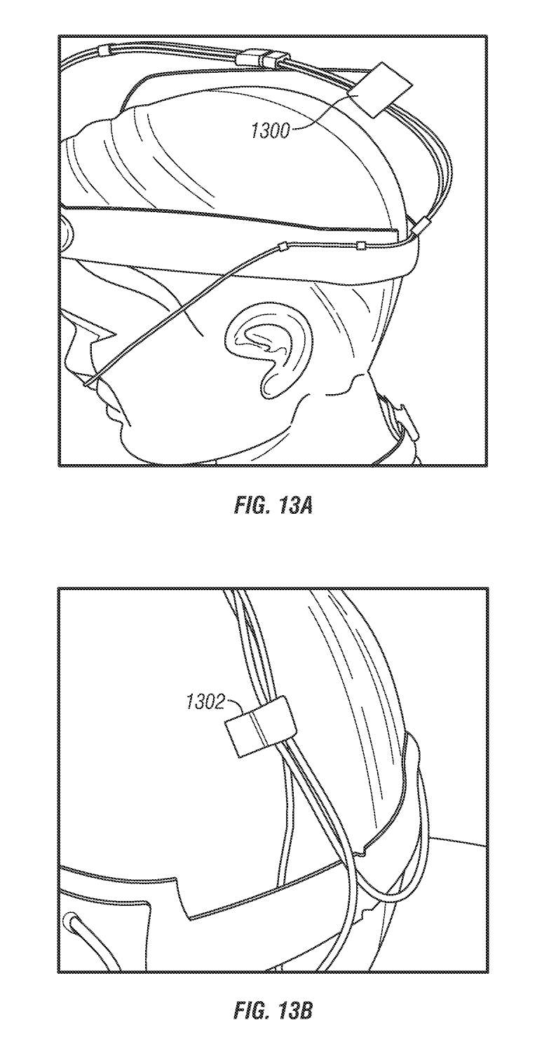

One example of a security label is shown in FIG. 12. Security label 1200 may be frangible and may include an adhesive portion or side that is covered by a release liner, which may be removed to expose the adhesive portion. After cable 1008 is connected to physiological monitoring system 1002, security label 1200, with the release liner removed, may be placed on both a micro-USB end 1016 of cable 1008 and physiological monitoring system 1002. As shown in FIGS. 13A and 13B, security labels 1300 and 1302 may be placed at other locations on cable 1008 to secure cable 1008 to other components of physiological monitoring system 1002 (e.g., an airflow sensor line). Nylon clips may also be used in addition or as an alternative to security label 1200 to secure cable 1008 to physiological monitoring system 1002.

If security label 1200 is removed after being affixed to cable 1008 and physiological monitoring system 1002, signs of tampering may be detected. For example, an adhesive residue may be left on cable 1008 and/or physiological monitoring system 1002 where security label 1200 was applied and then removed. Alternatively, once removed, security label 1200 may change in appearance and/or include a “void” message on security label 1200. These or other signs of tampering that may be evidenced on cable 1008 or physiological monitoring system 1002 may be detected by a clinician administering a home study, and the result of such home study may be void at the discretion of the clinician.

At step 1106, the physiological monitoring system is formatted. In system 1000, formatting of physiological monitoring system 1002 may be performed by a clinician to prepare physiological monitoring system 1002 for a new study and/or a new patient. Following or during the format of physiological monitoring system 1002, a unique identifier associated with receiver 1012 of chain of custody device 1010 is collected and stored in a data storage device such as memory 1004. Formatting also allows the clinician to indicate whether chain of custody device 1010 is required for a particular study. In cases where chain of custody device 1010 is not necessary, the clinician may format physiological monitoring system 1002 such that a connection to chain of custody device 1010 is not required in order for physiological monitoring system 1002 to perform its data collection operations.

At step 1108, the patient is provided with the physiological monitoring system. In reference to system 1000, this step involves providing the patient with physiological monitoring system 1002 that has already been formatted with cable 1008 attached. The patient may leave the office of the physician, but chain of custody device 1010 must be worn at all times. Upon returning to the office of the clinician, strap 1014 is removed by the clinician. The clinician may inspect chain of custody device 1010, cable 1008, and physiological monitoring system 1002 for signs of tampering. If any sign of tampering is detected, such as tearing of strap 1014 or a corrupted or removed security label, such as security label 1200 shown in FIG. 1200, the clinician may choose to void the result of the study.

### E. CONCLUSION

While various embodiments of the present invention have been described above, it should be understood that they have been presented by way of example only, and not limitation. For example, although certain embodiments of the present invention have been described in reference to a home sleep study, persons skilled in the relevant art(s) will readily appreciate that many other studies may benefit from the present invention. Furthermore, these studies may involve tasks other than physiological data collection, for example, administration of treatment.

It will be apparent to persons skilled in the relevant art(s) that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. A chain of custody device, comprising:
a strap for attaching to a body part of a patient;
a receiver connected to the strap that is operable to be connected to a physiological monitoring system via a cable, the receiver storing an identifier that is readable by the physiological monitoring system and usable by the physiological monitoring system to verify that the physiological monitoring system is connected to a correct chain of custody device; and
a security means for preventing undetected removal of the receiver.

2. The chain of custody device of claim 1, wherein the receiver is removably connected to the strap; and wherein the security means is also for preventing undetected removal of the receiver from the strap.

3. The chain of custody device of claim 1, wherein the receiver is removably connected to the strap via insertion of the strap into two openings on opposite ends of the receiver.

4. The chain of custody device of claim 1, wherein the security means comprises a snap rivet.

5. The chain of custody device of claim 1, wherein the strap comprises a hook and loop fastener.

6. The chain of custody device of claim 5, wherein the security means comprises a security tag having an adhesive portion that is looped through openings on the strap and sealed over the hook and loop fastener.

7. The chain of custody device of claim 5, wherein the strap comprises a napped cloth surface that facilitates the sealing of the hook and loop fastener.

8. The chain of custody device of claim 1, wherein the strap comprises a cloth inner surface to prevent irritation of the body of the patient.

9. The chain of custody device of claim 1, wherein the strap comprises a safety feature that facilitates breaking apart of the strap when stretched.

10. The chain of custody device of claim 9, wherein the safety feature comprises at least one perforated region that forms a breakage region in the strap.

11. The chain of custody device of claim 1, wherein the strap comprises a plurality of openings along a length of the strap, thereby permitting the strap to be adjustable.

12. The chain of custody of device of claim 1, wherein the strap is adapted to be worn around a neck of a patient.

13. The chain of custody device of claim 1, wherein the receiver is adapted to receive the cable and is operable to detect a presence of at least one of an extension cable between the receiver and the physiological monitoring system and a substitute cable between the receiver and the physiological monitoring system.

14. The chain of custody device of claim 1, wherein the receiver comprises an integrated circuit enclosed in a plastic molding.

15. A system comprising:
a physiological monitoring system; and
a chain of custody device, the chain of custody device comprising:
a strap for attaching to a body part of a patient;
a receiver that is operable to be connected to the physiological monitoring system via a cable, the receiver storing an identifier that is readable by the physiological monitoring system and usable by the physiological monitoring system to verify that the physiological monitoring system is connected to a correct chain of custody device; and
a security means for preventing undetected removal of the receiver.

16. The system of claim 15, wherein the physiological monitoring system is operable to cease physiological data collection and indicate an alarm upon detecting an incorrect chain of custody device.

17. The system of claim 16, wherein the alarm is an audible alarm.

18. The system of claim 15, wherein the physiological monitoring system is operable to cease physiological data collection and indicate an alarm upon the detection of a presence of an extension cable or a substitute cable connected to the receiver.

19. The system of claim 15, wherein the physiological monitoring system is operable to cease physiological data collection and indicate an alarm upon detecting an impaired connection with the receiver.

20. The system of claim 15, wherein the physiological monitoring system is operable to query the receiver periodically for the identifier from the receiver.

21. The system of claim 15, wherein the physiological monitoring system is operable to collect the identifier stored on the receiver for storage on a memory device accessible to the physiological monitoring system following a format of the physiological monitoring system.

22. A method for using a chain of custody device, comprising:
attaching a strap of a chain of custody device to a body part of patient, the chain of custody device including:
the strap,
a receiver that is operable to be connected to a physiological monitoring system via a cable, the receiver storing an identifier that is readable by the physiological monitoring system and usable by the physiological monitoring system to verify that the physiological monitoring system is connected to the correct chain of custody device, and
a security means for preventing undetected removal of the receiver;
securing the cable to the physiological monitoring system via a security label; and
providing the patient with the physiological monitoring system.

23. The method of claim 22, further comprising:
storing the identifier on a memory device accessible to the physiological monitoring system prior to providing the patient with the physiological monitoring system.
